(19) United States
(12) Patent Application Publication
(10) Pub. No.: US 2021/0353511 A1
(43) Pub. Date: Nov. 18, 2021

(54) GEL STICK COMPOSITIONS

(71) Applicant: CALUMET SPECIALTY PRODUCTS PARTNERS, L.P., Indianapolis, IN (US)

(72) Inventors: Mark A. VOELKER, Butler, PA (US); Angela VIERTEL, Parker, PA (US); Ryan B. CAMERON, Newtown, CT (US)

(21) Appl. No.: 17/284,994

(22) PCT Filed: Oct. 15, 2019
(86) PCT No.: PCT/US2019/056275

§ 371 Date: Apr. 13, 2021

Related U.S. Application Data
(60) Provisional application No. 62/746,213, filed on Oct. 16, 2018.

(51) Int. Cl. A61K 8/02; A61K 8/04; A61K 8/34; A61K 8/81; A61K 8/37; A61K 8/31; A61K 8/35; A61K 8/40; A61Q 17/04

(52) U.S. Cl. CPC A61K 8/0229; A61K 8/042; A61K 8/342; A61K 8/8117; A61K 8/375; A61K 2800/48; A61K 8/35; A61K 8/37; A61K 8/40; A61Q 17/04; A61K 8/31

(57) ABSTRACT

The present disclosure provides gel stick compositions. The gel stick compositions comprise i) one or more emollients; ii) one or more emulsifiers; iii) one or more block copolymers; iv) one or more co-gellants; and v) one or more antioxidants. Inclusion of a “co-gellant” to the described gel stick compositions provides several synergistic advantages of the resultant compositions.

### CROSS-REFERENCE TO RELATED APPLICATION

This application claims the benefit of and priority to U.S. Provisional Patent Application No. 62/746,213, filed Oct. 16, 2018, the entire disclosure of which is incorporated herein by reference.

### BACKGROUND AND SUMMARY OF THE INVENTION

Numerous gel compositions are known in the art. Gel compositions have proven to be useful in a wide variety of applications, such as in cosmetic, health and beauty, home care, and industrial applications. Although gel compositions in liquid, semi-solid, and gel formulations are currently utilized for many purposes, a solid gel “stick” formulation is highly desirable.

Accordingly, the present disclosure provides gel stick compositions that provide technical advantages compared to other gel compositions known in the art. For instance, the gel stick compositions of the present disclosure include a “co-gellant” that provides several synergistic advantages of the resultant compositions. First, inclusion of the co-gellant in the gel stick compositions can reduce the temperature that is required for gel formation. Generally, non-stick formulations of gel compositions are typically made at temperature between 240-260° F.

Second, inclusion of the co-gellant in the gel stick compositions can reduce the melting point (Mp) of the gelled system, which facilitates post-differentiation from a common formulation base. A lower melting point of the resulting gel stick compositions can advantageously save energy, while preserving the added ingredients and the subsequent differentiation stage of the final product. Gel compositions formed without the inclusion of a co-gellant can possess a melting point of approximately 230-266° F. Importantly, the melting point of the gelled system can be tailored to improve the structure and payout of the gel stick compositions to achieve compositions that possess a texture that is less spongy or rubbery in nature.

Furthermore, the co-gellant reduces the amount of other ingredients that are required for inclusion in the gel compositions, such as other co-gellants (e.g., amino acid dialkylamides) or block copolymers. In turn, this further results in a less expensive manner to produce the gel stick compositions.

Finally, the inclusion of co-gellant in the gel stick compositions results in an improvement in the aesthetic, structure, and performance of the gelled system. For example, the clarity, stability of final product, and ingredient transfer/payout are enhanced following inclusion of the co-gellant. In some formulations, advantageous properties of the resultant products such as lower hardness, lower friction of application, and lower resistance upon application are observed.

The following numbered embodiments are contemplated and are non-limiting:

1. A gel stick composition comprising:
i) one or more emollients;
ii) one or more emulsifiers;
iii) one or more block copolymers;
iv) one or more co-gellants; and
v) one or more antioxidants.

2. The gel stick composition of clause 1, wherein the one or more emollients is selected from the group consisting of fatty alkane lipids, fatty ester lipids, fatty acid esters, and polymeric alkane lipids.

3. The gel stick composition of clause 1 or clause 2, wherein the one or more emollients is present at about 40% to about 90% (w/w), or wherein the one or more emollients is present at about 70% to about 90% (w/w).

4. The gel stick composition of any one of clauses 1 to 3, wherein the one or more emollients is selected from the group consisting of hyaluronic acid, natural oils, essential oils, squalane, liponate (C12-C15 Alkyl benzoate), panalane (Hydrogenated Polyisobutene), mineral oil, isohexadecane, isododecane, hydrogenated poly (C6-C14 olefin), and Dermol BS (Butyl stearate).

5. The gel stick composition of any one of clauses 1 to 4, wherein the one or more emulsifiers is a fatty alcohol.

6. The gel stick composition of any one of clauses 1 to 5, wherein the one or more emulsifiers is present at about 3% to about 15% (w/w), or wherein the one or more emulsifiers is present at about 3% to about 40% (w/w).

7. The gel stick composition of any one of clauses 1 to 6, wherein the one or more emulsifiers is Uno Alkanol (Isostearyl alcohol).

8. The gel stick composition of any one of clauses 1 to 7, wherein the one or more block copolymers is selected from the group consisting of a diblock polymer, a triblock polymer, a star polymer, and combinations thereof.

9. The gel stick composition of any one of clauses 1 to 8, wherein the one or more block copolymers is selected from the group consisting of Kraton® G 1702, Kraton ® G 1701, Kraton® G 1780, Kraton® G 1650, Kraton® G 1652, Kraton® D 1101, Kraton® D 1102, Kraton®D 1133, Kraton® G1901, Kraton®D1160, and combinations thereof.

10. The gel stick composition of any one of clauses 1 to 8, wherein the one or more block copolymers is selected from the group consisting of Kraton® G 1726, Kraton® G 1643 ERS, Kraton® G 1648, Kraton® MD 6953, and combinations thereof.

11. The gel stick composition of any one of clauses 1 to 10, wherein the one or more block copolymers is present at about 0.01% to about 15%.

12. The gel stick composition of any one of clauses 1 to 11, wherein the one or more block copolymers is selected from the group consisting of Kraton® G 1650, Kraton® G 1702, or a combination thereof.

13. The gel stick composition of any one of clauses 1 to 12, wherein the one or more block copolymers is selected from the group consisting of Kraton® G 1726, Kraton® G 1643 ERS, or a combination thereof.

14. The gel stick composition of any one of clauses 1 to 13, wherein the one or more co-gellants is an amino acid dialkylamide.

15. The gel stick composition of any one of clauses 1 to 14, wherein the one or more co-gellants is present at about 0.01% to about 5% (w/w).

16. The gel stick composition of any one of clauses 1 to 15, wherein the one or more co-gellants is selected from the group consisting of GP-1 (dibutyl lauroylglutamide), EB-21 (dibutyl ethylhexanoyl glutamide), or a combination thereof.

17. The gel stick composition of any one of clauses 1 to 16, wherein the one or more antioxidants is Tinogard.

18. The gel stick composition of any one of clauses 1 to 17, wherein the one or more antioxidants is present at about 0.001% to about 1% (w/w).

19. The gel stick composition of any one of clauses 1 to 18 further comprising a clarifying component.

20. The gel stick composition of clause 19, wherein the clarifying component is selected from the group consisting of a structuring agent, a solubilizer, and a surfactant.

21. The gel stick composition of clause 19, wherein the clarifying component is a structuring agent.

22. The gel stick composition of clause 21, wherein the structuring agent is isostearic acid.

23. The gel stick composition of clause 22, wherein the isostearic acid is present at about 0.001% to about 5% (w/w).

24. The gel stick composition of clause 22, wherein the isostearic acid is present at about 1% to about 5% (w/w).

25. The gel stick composition of clause 22, wherein the isostearic acid is present at about 2% to about 4% (w/w).

26. The gel stick composition of clause 22, wherein the isostearic acid is present at about 3% (w/w).

27. The gel stick composition of clause 22, wherein the isostearic acid is present at about 3.5% (w/w).

28. The gel stick composition of clause 22, wherein the isostearic acid is present at about 4% (w/w).

29. The gel stick composition of any one of clauses 1 to 28 further comprising a processing component.

30. The gel stick composition of clause 29, wherein the processing component is selected from the group consisting of caprylic triglycerides and capric triglycerides.

31. A gel stick composition comprising:
i) a fatty alkane lipid, wherein the fatty alkane lipid is squalane;
ii) a fatty acid ester, wherein the fatty acid ester is Dermol BS (Butyl stearate);
iii) a fatty alcohol, wherein the fatty alcohol is Uno Alkanol (Isostearyl alcohol);
iv) a first block copolymer, wherein the first block copolymer is Kraton G 1650;
v) a second block copolymer, wherein the second block copolymer is Kraton G 1702;
vi) an amino acid dialkylamide co-gellant, wherein the amino acid dialkylamide co-gellant is GP-1 (dibutyl lauroylglutamide); and
vii) an antioxidant, wherein the antioxidant is Tinogard.

32. The gel stick composition of clause 31, wherein
i) the squalane is present at about 80.45% (w/w);
ii) the Dermol BS (Butyl stearate) is present at about 6% (w/w);
iii) the Uno Alkanol (Isostearyl alcohol) is present at about 6% (w/w);
iv) the Kraton G 1650 is present at about 5% (w/w);
v) the Kraton G 1702 is present at about 1.5% (w/w);
vi) the GP-1 (dibutyl lauroylglutamide) is present at about 1% (w/w); and
vii) the Tinogard is present at about 0.05% (w/w).

33. A gel stick composition comprising:
i) a fatty ester lipid, wherein the fatty alkane lipid is liponate (C12-C15 Alkyl benzoate);
ii) a fatty acid ester, wherein the fatty acid ester is Dermol BS (Butyl stearate);
iii) a fatty alcohol, wherein the fatty alcohol is Uno Alkanol (Isostearyl alcohol);
iv) a first block copolymer, wherein the first block copolymer is Kraton G 1650;
v) a second block copolymer, wherein the second block copolymer is Kraton G 1702;
vi) an amino acid dialkylamide co-gellant, wherein the amino acid dialkylamide co-gellant is GP-1 (dibutyl lauroylglutamide); and
vii) an antioxidant, wherein the antioxidant is Tinogard.

34. The gel stick composition of clause 33, wherein
i) the liponate (C12-C15 Alkyl benzoate) is present at about 76.55% (w/w);
ii) the Dermol BS (Butyl stearate) is present at about 6% (w/w);
iii) the Uno Alkanol (Isostearyl alcohol) is present at about 6% (w/w);
iv) the Kraton G 1650 is present at about 6.3% (w/w);
v) the Kraton G 1702 is present at about 2.8% (w/w);
vi) the GP-1 (dibutyl lauroylglutamide) is present at about 2.3% (w/w); and
vii) the Tinogard is present at about 0.05% (w/w).

35. A gel stick composition comprising:
i) a polymeric alkane lipid, wherein the panalane (Hydrogenated Polyisobutene);
ii) a fatty acid ester, wherein the fatty acid ester is Dermol BS (Butyl stearate);
iii) a fatty alcohol, wherein the fatty alcohol is Uno Alkanol (Isostearyl alcohol);
iv) a first block copolymer, wherein the first block copolymer is Kraton G 1650;
v) a second block copolymer, wherein the second block copolymer is Kraton G 1702;
vi) an amino acid dialkylamide co-gellant, wherein the amino acid dialkylamide co-gellant is GP-1 (dibutyl lauroylglutamide); and
vii) an antioxidant, wherein the antioxidant is Tinogard.

36. The gel stick composition of clause 35, wherein
i) the panalane (Hydrogenated Polyisobutene) is present at about 80.45% (w/w);
ii) the Dermol BS (Butyl stearate) is present at about 6% (w/w);
iii) the Uno Alkanol (Isostearyl alcohol) is present at about 6% (w/w);
iv) the Kraton G 1650 is present at about 5% (w/w);
v) the Kraton G 1702 is present at about 1.5% (w/w);
vi) the GP-1 (dibutyl lauroylglutamide) is present at about 1% (w/w); and
vii) the Tinogard is present at about 0.05% (w/w).

37. A gel stick composition comprising hydrogenated polyisobutene, butyl stearate, isostearyl alcohol, hydrogenated styrene/butadiene copolymer, octyl dodecanol, dibutyl lauroyl glutamide, dibutyl ethylhexanoyl glutamide, and pentaerythriyl tetra-di-t-butyl hydroxyhydrocinnamate.

38. The gel stick composition of clause 37 further comprising butyl methoxydibenzoylmethane.

39. The gel stick composition of clause 37 or clause 38 further comprising homosalate.

40. The gel stick composition of any one of clauses 37 to 39 further comprising ethylhexyl salicylate.

41. The gel stick composition of any one of clauses 37 to 40 further comprising octocrylene.

42. A gel stick composition comprising Squalane, Butyl Stearate, Isostearyl Alcohol, Hydrogenated Styrene/Butadiene Copolymer, Octyl Dodecanol, Dibutyl Lauroyl Glutamide, Dibutyl Ethylhexanoyl Glutamide, and Pentaerythrityl Tetra-Di-t-Butyl Hydroxyhydrocinnamate.

43. The gel stick composition of clause 42 further comprising Zinc Oxide, C13-15 Alkane & Polyglyceryl-3 polyricinoleate, Sorbitan Isostearate, and Triethoxycaprylylsilane.

44. The gel stick composition of clause 42 or clause 43 further comprising Titanium Dioxide, C13-15 Alkane & Polyglyceryl-3 polyricinoleate, Sorbitan Isostearate, Silica, and Triethoxycaprylylsilane.

### BRIEF DESCRIPTION OF THE DRAWINGS

FIG. 1 is a schematic illustrating the chain structure of a diblock copolymer used in embodiments of the present disclosure.

FIG. 2 is a schematic illustrating the chain structure of a triblock copolymer used in embodiments of the present disclosure.

FIG. 3 is a schematic illustrating the chain structure of a radial polymer used in embodiments of the present disclosure.

FIG. 4 is a schematic illustrating the chain structure of a star polymer used in embodiments of the present disclosure.

FIGS. 5A-5B are schematics illustrating the chain structure of multi-block copolymers used in embodiments of the present disclosure.

FIG. 6 shows the gel stick raw material for the gel stick composition described herein.

FIG. 7 shows the gel stick product for the gel stick composition described herein.

FIG. 8 shows the texture analyzer utilized for hardness testing.

FIG. 9 shows hardness parameters for three marketed products comprising wax-thickened silicone (SB, BG, and NK) compared to exemplary sunscreen products formulated according to the present disclosure (VS-M, VS-ME, and VS-SQ).

FIG. 10 shows the texture analyzer utilized for friction and transfer rate testing.

FIG. 11 shows friction parameters for three marketed products comprising wax-thickened silicone (SB, BG, and NK) compared to exemplary sunscreen products formulated according to the present disclosure (VS-M, VS-ME, and VS-SQ).

FIG. 12 shows transfer rate parameters for three marketed products comprising wax-thickened silicone (SB, BG, and NK) compared to exemplary sunscreen products formulated according to the present disclosure (VS-M, VS-ME, and VS-SQ).

Various embodiments of the invention are described herein as follows. In one embodiment described herein, a gel stick composition is provided. The gel stick composition comprises i) one or more emollients; ii) one or more emulsifiers; iii) one or more block copolymers; iv) one or more co-gellants; and v) one or more antioxidants.

All percentages stated in the present disclosure are presumed to be weight/weight (w/w) percentages, unless indicated otherwise. Furthermore, weight/weight (w/w) percentages are presumed to add up to 100% in total. However, if the stated weight/weight (w/w) percentages add up to more than 100% in total, then the percentage(s) of one or more emollients may be reduced so that the total weight/weight (w/w) percentage adds up to 100%.

As used herein, the term “gel stick composition” indicates that the gel composition is in solid form, such as formulation as a stick product.

In some embodiments, the one or more emollients is selected from the group consisting of fatty alkane lipids, fatty ester lipids, fatty acid esters, and polymeric alkane lipids. In various embodiments, the one or more emollients is present at about 40% to about 90% (w/w). In various embodiments, the one or more emollients is present at about 70% to about 90% (w/w). In certain embodiments, the one or more emollients is hyaluronic acid. In other embodiments, the one or more emollients is a natural oil. In yet other embodiments, the one or more emollients is an essential oil.

In certain embodiments, the one or more emollients is selected from the group consisting of hyaluronic acid, natural oils, essential oils, squalane, liponate (C12-C15 Alkyl benzoate), panalane (Hydrogenated Polyisobutene), mineral oil, isohexadecane, isododecane, hydrogenated poly (C6-C14 olefin), and Dermol BS (Butyl stearate).

In some embodiments, the one or more emulsifiers is a fatty alcohol. In various embodiments, the one or more emulsifiers is present at about 3% to about 15% (w/w). In various embodiments, the one or more emulsifiers is present at about 3% to about 40% (w/w). In certain embodiments, the one or more emulsifiers is Uno Alkanol (Isostearyl alcohol).

In some embodiments of the invention, a block copolymer capable of forming a three-dimensional network through physical cross-linking is used as the gelling agent. Suitable block copolymers include at least one rigid block and one elastomeric block. The rigid blocks of a block copolymer form rigid domains through which physical cross-linking may occur. The physical cross-linking via these rigid domains yields a continuous three-dimensional network. In the presence of heat, shear, or solvent, the rigid domains soften and permit flow. Upon cooling, removal of shear, or solvent evaporation, the rigid domains reform and harden, locking the elastomeric network in place. Preferably, suitable block copolymers include diblock copolymers, triblock copolymers, radial polymers, star polymers, multi-block copolymers, and mixtures thereof.

FIG. 1 illustrates the typical chain structure of a diblock copolymer. The polymer chain of the diblock copolymer includes two blocks: a rigid block and an elastomeric block. The rigid block is represented by diamonds. The elastomeric block is represented by circles. The rigid block typically is composed of polystyrene, polyethylene, polyvinylchloride, phenolics, and the like; the elastomeric block may be composed of, ethylene/butadiene copolymers, polyisoprene, polybutadiene, ethylene/propylene copolymers, ethylene-propylene/diene copolymers, and the like. As such, suitable diblock copolymers include, but are not limited to, styrene-ethylene/propylene copolymers, styrene-ethylene/butadiene copolymers, styrene-isoprene copolymers, styrene-butadiene copolymers.

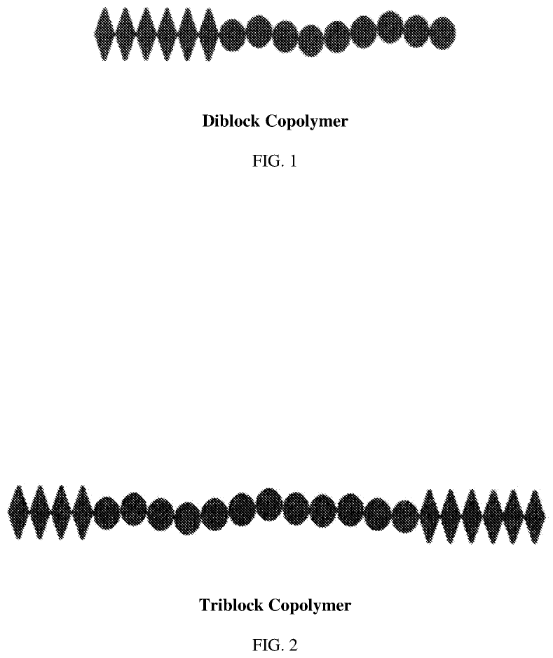

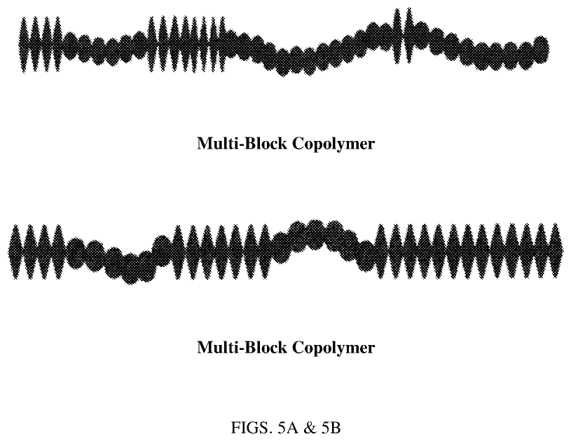

FIG. 2 illustrates the chain structure of a triblock copolymer. As illustrated in FIG. 2, each polymer chain includes two rigid blocks at either end and a middle block which is elastomeric. This is a preferred triblock copolymer structure, although a triblock copolymer with two elastomeric end blocks and a rigid middle block also can be used. Suitable triblock copolymers include, but are not limited to, styrene-ethylene/propylene-styrene copolymers, styrene-ethylene/butadiene-styrene copolymers, styrene-isoprene-styrene copolymers, and styrene-butadiene-styrene copolymers. Multi-block copolymers are similar to diblock copolymers or triblock copolymers, except that the multiple block copolymers include additional elastomeric blocks and/or rigid blocks as illustrated in FIGS. 5A-5B.

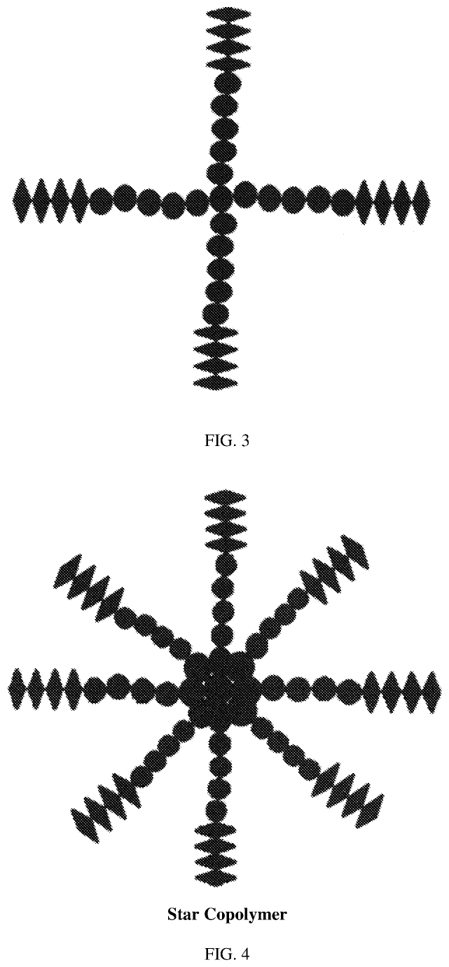

In addition to the linear chain structure, branched homopolymers or copolymers also may be used. FIG. 3 and FIG. 4 illustrate the chain structure of a radial polymer and a star polymer. It should be noted that one or more functional groups may be grafted onto the chain of any of the aforementioned polymers. In other words, any of the above polymers may be modified by grafting. Suitable functional groups for grafting depend on the desired properties. For example, one or more ester groups, silane groups, silicon-containing groups, maleic anhydride groups, acrylamide groups, and acid groups may be grafted. In addition to grafting, the above polymers may be hydrogenated to reduce unsaturation before they are used as gelling agents.

Numerous commercially available block copolymers may be used in embodiments of the invention. For example, various grades of copolymers sold under the trade name of Kraton® from Shell Chemical Company can be used as a gelling agent. In addition, copolymers sold under the trade name of Vector® available from Dexco® and Septon® from Kuraray also may be used. U.S. Pat. Nos. 5,221,534, 5,578,089, and 5,879,694 disclose block copolymers which may be used in embodiments of the invention, and the disclosures of these three patents are incorporated by reference in their entirety herein.

Table 1 lists some commercially available block copolymers which may be used in embodiments of the invention. It is noted that additional suitable block copolymers may include, but are not limited to, polystyrene/polyester, polyether/polyamide, polyether/polyester, polyester/polyamide, polyether/polyurethane, polyester/polyurethane, poly(ethylene oxide)/poly(propylene oxide), nylon/rubber, and polysiloxane/polycarbonate.

TABLE 1

| | | Polystyrene | |
|---|---|---|---|
| Copolymer | Block Type | Content (%) | Comment |
| Kraton ® | SEP | 28 | hydronated diblock |
| G 1702 | | | |
| Kraton ® | SEP | 37 | |
| G 1701 | | | |
| Kraton ® | SEP | 7 | Star polymer |
| G 1780 | | | |
| Kraton ® | SEBS | 30 | hydronated triblock |
| G 1650 | | | |
| Kraton ® | SEBS | 30 | hydronated triblock |
| G 1652 | | | |
| Kraton ® | SEBS + SEP | 31 | triblock and diblock |
| D 1101 | | | mixture (85:15) |
| Kraton ® | SEBS + SEP | 28 | triblock + diblock |
| D 1102 | | | (85:15) |
| Kraton ® | SEBS + SEP | 35 | triblock + diblock |
| D 1133 | | | (66:34) |
| Kraton ® | SEBS | 30 | triblock (hydronated |
| FG 1901 | | | anf functionally |
| | | | grafted with 1.7% |
| | | | of maleic anhydride |
| Septon ® | SEP | 35 | hydronated diblock |
| 1001 | | | |
| Vector ® | SEP | 30 | Unsaturated diblock |
| 6030 | | | |
| Vector ® | SBS | 29 | Unsaturated triblock |
| 8550 | | | |
| Vector ® | SBS | 31 | Unsaturated triblock |
| 2518P | | | |
| Solprene ® | SB | 40 | Unsaturated diblock |
| 1430 | | | |

NOTE:
SEP denotes to Styrene/ethylene/propylene copolymers
SEBS denotes to stytene/ethylene/bytolene/styrene copolymers
SB denotes to styrene/butadiene copolymers
SBS denotes to styrene-butadiene-styrene copolymers

In some embodiments, the one or more block copolymers is selected from the group consisting of a diblock polymer, a triblock polymer, a star polymer, and combinations thereof.

In various embodiments, the one or more block copolymers is selected from the group consisting of Kraton® G 1702, Kraton® G 1701, Kraton® G 1780, Kraton® G 1650, Kraton® G 1652, Kraton® D 1101, Kraton® D 1102, Kraton®D 1133, Kraton® G1901, Kraton® D1160, and combinations thereof.

In various embodiments, the one or more block copolymers is selected from the group consisting of Kraton® G 1726, Kraton® G 1643 ERS, Kraton® G 1648, Kraton® MD 6953, and combinations thereof.

In certain embodiments, the one or more block copolymers is present at about 0.01% to about 15%. In yet other embodiments, the one or more block copolymers is selected from the group consisting of Kraton® G 1650, Kraton® G 1702, or a combination thereof. In other embodiments, the one or more block copolymers is selected from the group consisting of Kraton® G 1726, Kraton® G 1643 ERS, or a combination thereof.

In some embodiments, the one or more co-gellants is an amino acid dialkylamide. In various embodiments, the one or more co-gellants is present at about 0.01% to about 5% (w/w).

In certain embodiments, the one or more co-gellants is selected from the group consisting of GP-1 (dibutyl lauroyl glutamide), EB-21 (dibutyl ethylhexanoyl glutamide), or a combination thereof. The co-gellant GP-1 is also known as dibutyl lauroyl glutamide and has the following structure:

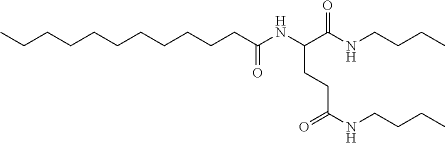 [SMILES: CCCCCCCCCCCC(=O)NC(CCC(=O)NCCCC)C(=O)NCCCC]

The co-gellant EB-21 is also known as dibutyl ethylhexanoyl glutamide and has the following structure:

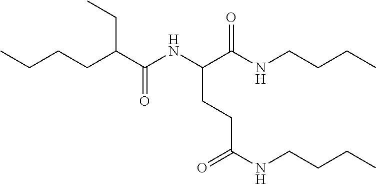 [SMILES: CCCCNC(=O)CCC(NC(=O)C(CC)CCCC)C(=O)NCCCC]

In illustrative embodiments, a premix known as AJK-OD2046 (Ajinomoto Co., Inc., Tokyo, Japan) can be utilized as a co-gellant. AJK-OD2046 comprises GP-1, EB-21, and a polar oil octyldodecanol. AJK-OD2046 comprises 20% active of the co-gellants together in a ratio of 60:40 (GP-1:EB-21).

In some embodiments, the one or more antioxidants is Tinogard. In certain embodiments, the one or more antioxidants is present at about 0.001% to about 1% (w/w).

In certain aspects, the gel stick composition further comprises a clarifying component. In illustrative embodiments, the clarifying component is selected from the group consisting of a structuring agent, a solubilizer, and a surfactant.

In some aspects, the clarifying component is a structuring agent. In various embodiments, the structuring agent is isostearic acid. In some embodiments, the isostearic acid is present at about 0.001% to about 5% (w/w). In other embodiments, the isostearic acid is present at about 1% to about 5% (w/w). In yet other embodiments, the isostearic acid is present at about 2% to about 4% (w/w). In other embodiments, the isostearic acid is present at about 3% (w/w). In yet other embodiments, the isostearic acid is present at about 3.5% (w/w). In other embodiments, the isostearic acid is present at about 4% (w/w).

In various aspects, the gel stick composition further comprises a processing component. In illustrative embodiments, the processing component is selected from the group consisting of caprylic triglycerides and capric triglycerides. Without being bound by any theory, the processing component may be included in the gel stick compositions of the present disclosure in order to lower the melting/processing temperature of the composition, to improve the ease of clean-up, and the like.

In one aspect, a gel stick composition is provided, wherein the gel stick composition comprises i) a fatty alkane lipid, wherein the fatty alkane lipid is squalane; ii) a fatty acid ester, wherein the fatty acid ester is Dermol BS (Butyl stearate); iii) a fatty alcohol, wherein the fatty alcohol is Uno Alkanol (Isostearyl alcohol); iv) a first block copolymer, wherein the first block copolymer is Kraton G 1650; v) a second block copolymer, wherein the second block copolymer is Kraton G 1702; vi) an amino acid dialkylamide co-gellant, wherein the amino acid dialkylamide co-gellant is GP-1 (Dibutyl lauroyl glutamide); and vii) an antioxidant, wherein the antioxidant is Tinogard.

In a further embodiment of this gel stick composition, i) the squalane is present at about 80.45% (w/w); ii) the Dermol BS (Butyl stearate) is present at about 6% (w/w); iii) the Uno Alkanol (Isostearyl alcohol) is present at about 6% (w/w); iv) the Kraton G 1650 is present at about 5% (w/w); v) the Kraton G 1702 is present at about 1.5% (w/w); vi) the GP-1 (Dibutyl lauroyl glutamide) is present at about 1% (w/w); and vii) the Tinogard is present at about 0.05% (w/w).

In another aspect, a gel stick composition is provided, wherein the gel stick composition comprises i) a fatty ester lipid, wherein the fatty alkane lipid is liponate (C12-C15 Alkyl benzoate); ii) a fatty acid ester, wherein the fatty acid ester is Dermol BS (Butyl stearate); iii) a fatty alcohol, wherein the fatty alcohol is Uno Alkanol (Isostearyl alcohol); iv) a first block copolymer, wherein the first block copolymer is Kraton G 1650; v) a second block copolymer, wherein the second block copolymer is Kraton G 1702; vi) an amino acid dialkylamide co-gellant, wherein the amino acid dialkylamide co-gellant is GP-1 (Dibutyl lauroyl glutamide); and vii) an antioxidant, wherein the antioxidant is Tinogard.

In a further embodiment of this gel stick composition, i) the liponate (C12-C15 Alkyl benzoate) is present at about 76.55% (w/w); ii) the Dermol BS (Butyl stearate) is present at about 6% (w/w); iii) the Uno Alkanol (Isostearyl alcohol) is present at about 6% (w/w); iv) the Kraton G 1650 is present at about 6.3% (w/w); v) the Kraton G 1702 is present at about 2.8% (w/w); vi) the GP-1 (Dibutyl lauroyl glutamide) is present at about 2.3% (w/w); and vii) the Tinogard is present at about 0.05% (w/w).

In yet another aspect, a gel stick composition is provided, wherein the gel stick composition comprises i) a polymeric alkane lipid, wherein the panalane (Hydrogenated Polyisobutene); ii) a fatty acid ester, wherein the fatty acid ester is Dermol BS (Butyl stearate); iii) a fatty alcohol, wherein the fatty alcohol is Uno Alkanol (Isostearyl alcohol); iv) a first block copolymer, wherein the first block copolymer is Kraton G 1650; v) a second block copolymer, wherein the second block copolymer is Kraton G 1702; vi) an amino acid dialkylamide co-gellant, wherein the amino acid dialkylamide co-gellant is GP-1 (Dibutyl lauroyl glutamide); and vii) an antioxidant, wherein the antioxidant is Tinogard.

In a further embodiment of this gel stick composition, i) the panalane (Hydrogenated Polyisobutene) is present at about 80.45% (w/w); ii) the Dermol BS (Butyl stearate) is present at about 6% (w/w); iii) the Uno Alkanol (Isostearyl alcohol) is present at about 6% (w/w); iv) the Kraton G 1650 is present at about 5% (w/w); v) the Kraton G 1702 is present at about 1.5% (w/w); vi) the GP-1 (Dibutyl lauroyl glutamide) is present at about 1% (w/w); and vii) the Tinogard is present at about 0.05% (w/w).

In yet another aspect, a gel stick composition is provided, wherein the gel stick composition comprises hydrogenated polyisobutene, butyl stearate, isostearyl alcohol, hydrogenated styrene/butadiene copolymer, octyl dodecanol, dibutyl lauroyl glutamide, dibutyl ethylhexanoyl glutamide, and pentaerythriyl tetra-di-t-butyl hydroxyhydrocinnamate.

In a further embodiment of this gel stick composition, the gel stick composition further comprises butyl methoxydibenzoylmethane. In a further embodiment of this gel stick composition, the gel stick composition further comprises homosalate. In a further embodiment of this gel stick composition, the gel stick composition further comprises ethylhexyl salicylate. In a further embodiment of this gel stick composition, the gel stick composition further comprises octocrylene.

In yet another aspect, a gel stick composition is provided, wherein the gel stick composition comprises Squalane, Butyl Stearate, Isostearyl Alcohol, Hydrogenated Styrene/Butadiene Copolymer, Octyl Dodecanol, Dibutyl Lauroyl Glutamide, Dibutyl Ethylhexanoyl Glutamide, and Pentaerythrityl Tetra-Di-t-Butyl Hydroxyhydrocinnamate.

In a further embodiment of this gel stick composition, the gel stick composition further comprises Zinc Oxide, C13-15 Alkane & Polyglyceryl-3 polyricinoleate, Sorbitan Isostearate, and Triethoxycaprylylsilane.

In a further embodiment of this gel stick composition, the gel stick composition further comprises Titanium Dioxide, C13-15 Alkane & Polyglyceryl-3 polyricinoleate, Sorbitan Isostearate, Silica, and Triethoxycaprylylsilane.

The entire disclosures of U.S. Pat. No. 6,881,776, issued on Apr. 19, 2005, and of U.S. Pat. No. 9,339,446, issued on May 17, 2016, are hereby incorporated herein by reference in their entirety.

### EXAMPLE 1

### Process for Making Gel Stick Composition

In the instant example, a process for making an exemplary gel stick composition is presented. First, the emollients and emulsifiers are combined and heated to a temperature of about 220° F. Thereafter, the co-gellant is added. The combination is then mixed, and heat is continued to be applied until the combination is dissolved.

After the combination is dissolved, the block copolymers and the antioxidant are added. Mixing is continued and heat is applied at about 270° F.-280° F. for approximately 5 hours, or until all ingredients are dissolved.

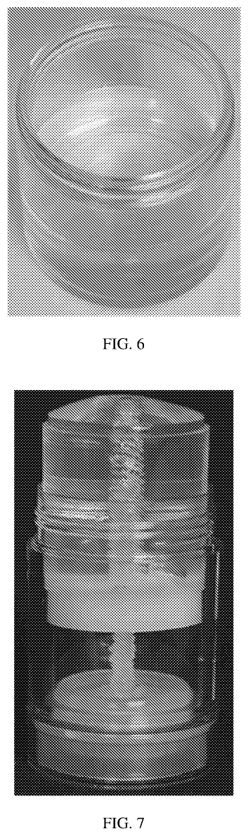

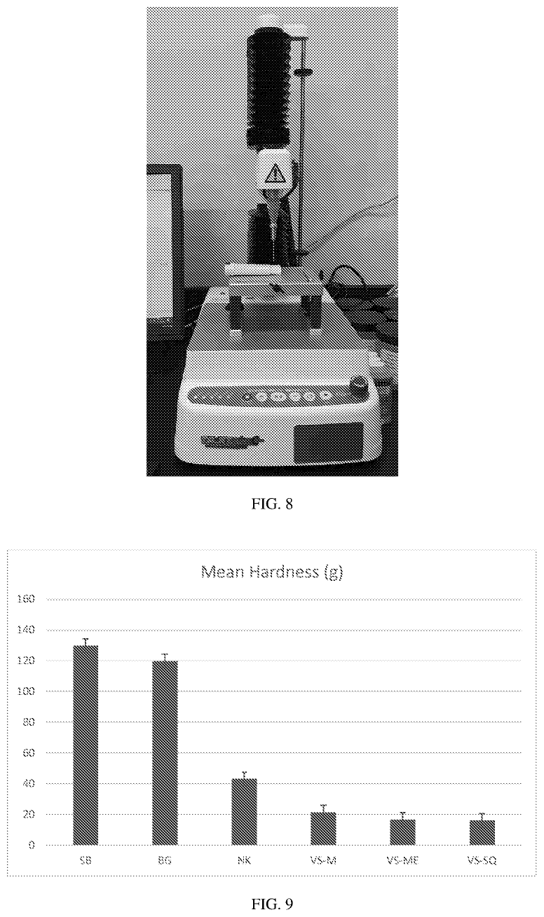

The dissolved combination is then cooled to room temperate in order to obtain the gel stick composition. FIG. 6 shows the gel stick raw material of the gel stick composition. FIG. 7 shows the gel stick product of the gel stick composition.

Furthermore, Table 2 demonstrates various properties observed with exemplary gel stick compositions provided in the present disclosure.

TABLE 2

| | Base Lipid | | |
|---|---|---|---|
| | | C12 - C15 | Hydrogenated |
| Property | Squalane | Alkyl benzoate | Polyisobutene |
| Viscosity | 30.0 - 55.0 | 30.0 - 55.0 | 30.0 - 55.0 |
| @13° C. (cps) | | | |
| Specific Gravity | 0.8100 | 0.9300 | 0.8300 |
| @ 25° C. | | | |
| Flash Point | 220 | 185 | 140 |
| ASTM D-92 (C) | | | |

### EXAMPLE 2

### Process for Improving Clarity of Compositions

In the instant example, levels of exemplary co-gellants were varied in the oil blends to evaluate the clarity of the resultant compositions. The exemplary co-gellants GP-1 and EB-21 were tested.

Several composition blends were formulated comprising block copolymers, including 1.5% Kraton® G 1702 and 5% Kraton® G 1650 with varying levels of co-gellants GP-1 and EB-21. The visual appearance of the compositions was then characterized as follows: H=hazy, C=clear, S=slightly, P=precipitate, V=very. The results are shown in Table 3 below.

TABLE 3
Effect of co-gellants on clarity and melting point

| Product | GP-1 | EB-21 | Visual | Mp (° C.) |
|---|---|---|---|---|
| S1000T | 1 | 0 | H | 116 |
| Blend 4 | 0.5 | 0.5 | C | 117 |
| Blend 5 | 0.1 | 0.9 | SH, P | 132 |
| Blend 6 | 0 | 1 | VH | 137 |
| Blend 7 | 0 | 0.5 | SH | 121 |
| Blend 8 | 0.5 | 0 | SH | 104 |
| Blend 9 | 0.3 | 0.2 | SH | 99 |

As shown in Table 3, the co-gellant EB-21 was demonstrated to increase the melting point of the blends more than GP-1. The level of co-gellant was observed to affect both clarity and melting point of the resultant compositions. It may be desired to have a melting point at or below 100° C. to enable processing in some manufacturing equipment, and this factor may be balanced with other considerations in formulating the compositions.

### EXAMPLE 3

### Effect of Copolymer Levels in Absence of Co-Gellant in the Compositions

In the instant example, different levels of block copolymers were incorporated into oil blends to investigate the effect of thickening with co-polymers in the absence of co-gellants. Various levels of the exemplary block copolymer Kraton® G 1650 were incorporated into an oil blend consisting of approximately 85% hydrogenated polyisobutene, 6% butyl stearate, 6% isostearyl alcohol, and 0.05% Tinogard. The visual and textural properties of the resultant blends were characterized as follows: H=hazy, S=spongy, OB=oil bleed, V=very. The results are shown in Table 4 below.

TABLE 4
Effect of copolymer levels in absence of co-gellant

| G 1650 (%) | Mp (° C.) | Hardness (mm) | Observation |
|---|---|---|---|
| 3 | 66 | 314 | S, H, OB |
| 4 | 67 | 276 | S, H, OB |
| 5 | 67 | 238 | S, H, OB |
| 5.5 | 68 | 227 | VS, H, OB |
| 6.5 | 69 | 214 | VS, H, OB |

For the evaluation of cone penetrometer hardness, a smaller number indicates a harder/firmer product. Thus, Table 4 shows that resultant compositions were observed to be harder/firmer as the level of block copolymer was increased. However, the compositions remained spongy, hazy, and exhibited oil bleed with increasing block copolymer level. These observations suggest that inclusion of a suitable co-gellant may be necessary to achieve the desired structure, performance, and stability of the gel stick compositions. A comparison of the melting points shown in Table 3 above demonstrates that the co-gellants can increase the melting point of the gel stick compositions.

### EXAMPLE 4

### Effect of Kraton® G 1650 Copolymer Levels in Presence of Co-Gellant in the Compositions

The instant example evaluated optimal levels of the exemplary block copolymer Kraton® G 1650 for various co-gellant systems. Various levels of Kraton® G 1650 were incorporated into an oil blend comprising approximately 85% hydrogenated polyisobutene, 6% butyl stearate, 6% isostearyl alcohol, 0.25% GP-1, 0.25% EB-21, and 0.05% Tinogard. The visual and textural properties of the resultant blends were characterized as follows: C=clear, H=hazy, SH=slightly hazy, S=solid, SS=semisolid, SE=spreads easily, SR=slightly rubbery, R=rubbery. The results are shown in Table 5 below.

TABLE 5
Effect of Kraton 0 G 1650 copolymer levels in the presence of co-gellants

| G 1650 (%) | Mp (° C.) | Hardness (mm) | Observation |
|---|---|---|---|
| 0 | — | — | C, SS, SE |
| 1 | — | — | H, SS, SE |
| 2 | — | — | H, SS, SE |
| 3 | 103 | 168 | H, S, SE |
| 4 | 104 | 164 | SH, S, SR |
| 5 | 103 | 158 | SH, S, R |
| 5.5 | 103 | 153 | SH, S, R |
| 6 | 103 | 157 | C, S, R |

For the evaluation of cone penetrometer hardness, a smaller number indicates a harder/firmer product. Thus, Table 5 shows that resultant compositions were observed to be harder/firmer as the copolymer level was increased. As co-gellants are generally more expensive than copolymers, it may be advantageous to use the smallest efficacious level of co-gellants in the blends.

In the instant example, the Kraton® G 1650 block copolymer at 3% was an acceptable product in terms of hardness and transfer rate, but this exemplary product was hazy and not clear as would be aesthetically preferred. At concentrations of 4-6% Kraton® G 1650, the product became increasingly clear but also increasing rubbery. At a concentration of 6% Kraton® G 1650, the product is clear but too rubbery for generating a stick product that effectively transferred material to a surface upon application.

### EXAMPLE 5

### Effect of Kraton® G 1726 Copolymer Levels in Presence of Co-Gellant in the Compositions

The instant example evaluated optimal levels of the shorter chain length block copolymer Kraton® G 1726 for various co-gellant systems. Use of short-range aggregates in the gel stick compositions were contrasted to the longer entanglements created by longer chain length block polymers in other examples. Thus, various levels of Kraton® G 1726 were incorporated into an oil blend comprising approximately 85% hydrogenated polyisobutene, 6% butyl stearate, 6% isostearyl alcohol, (a: 0.25% GP-1, 0.25% EB-21 orb: 0.50% GP-1, 0.50% EB-21), and 0.05% Tinogard. The visual and textural properties of the resultant blends were characterized as follows: C=clear, H=hazy, SH=slightly hazy, S=solid, SS=semisolid, SE=spreads easily, SR=slightly rubbery, R=rubbery. The results are shown in Table 6 below.

TABLE 6
Effect of Kraton ® G 1726 copolymer levels in
the presence of co-gellants

| G 1726 (%) | Mp (° C.) | Hardness (mm) | Observation |
|---|---|---|---|
| 4^{a} | 102 | 179 | C, S, SE |
| 5^{a} | 102 | 174 | C, S, SR |
| 4^{b} | 117 | 147 | C, S, SE |

For the evaluation of cone penetrometer hardness, a smaller number indicates a harder/firmer product. Thus, Table 6 shows that although resultant compositions were observed to be slightly harder/firmer as the copolymer level was increased from 4 to 5%, the product became too rubbery. Table 6 demonstrates that increasing levels of co-gellant provides increased structuring of the gel stick compositions, without making them too rubbery, and maintained clarity. Therefore, the shorter chain length block copolymer Kraton® G 1726 was observed to provide an improved balance of clarity, structure, and ease of application for the resultant gel stick compositions.

### EXAMPLE 6

### Effect of Kraton® G 1643 ERS Copolymer Levels in Presence of Co-Gellant in the Compositions

The instant example evaluated optimal levels of Kraton® G 1643 Enhanced Rubber Segment (ERS) for various co-gellant systems. Thus, various levels of Kraton® G 1643 ERS were incorporated into an oil blend comprising approximately 85% hydrogenated polyisobutene, 6% butyl stearate, 6% isostearyl alcohol, (a: 0.25% GP-1, 0.25% EB-21 orb: 0.50% GP-1, 0.50% EB-21), and 0.05% Tinogard. The visual and textural properties of the resultant blends were characterized as follows: C=clear, H=hazy, SH=slightly hazy, S=solid, SS=semisolid, SE=spreads easily, SR=slightly rubbery, R=rubbery. The results are shown in Table 7 below.

TABLE 7
Effect of Kraton ® G 1643 ERS copolymer
levels in the presence of co-gellants

| G 1643 | | | |
|---|---|---|---|
| ERS (%) | Mp (° C.) | Hardness (mm) | Observation |
| 4^{a} | 102 | 176 | C, S, SE |
| 5^{a} | 104 | 153 | C, S, SR |
| 4^{b} | 117 | 144 | C, S, SE |

For the evaluation of cone penetrometer hardness, a smaller number indicates a harder/firmer product. Thus, Table 7 shows that although the resultant compositions became slightly harder/firmer as the copolymer level was increased from 4 to 5%, the product became too rubbery. Table 7 also demonstrates that increasing levels of co-gellant provides increased structuring of the gel stick compositions, without making them too rubbery, and maintained clarity. A concentration of 4% Kraton® G 1643 ERS resulted in a gel stick composition that was less structured compared to use of a concentration 4% Kraton® G 1726 but still was observed to have good overall properties.

### EXAMPLE 7

### Effect of Kraton® MD 1648 Copolymer Levels in Presence of Co-Gellant in the Compositions

The instant example evaluated optimal levels of Kraton® MD 1648 for various co-gellant systems. Thus, various levels of MD 1648 were incorporated into an oil blend comprising approximately 85% hydrogenated polyisobutene, 6% butyl stearate, 6% isostearyl alcohol, (a: 0.25% GP-1, 0.25% EB-21 orb: 0.50% GP-1, 0.50% EB-21), and 0.05% Tinogard. The visual and textural properties of the blends were characterized as follows: C=clear, H=hazy, SH=slightly hazy, S=solid, SS=semisolid, SE=spreads easily, SR=slightly rubbery, R=rubbery. The results are shown in Table 8 below.

TABLE 8
Effect of Kraton ® MD 1648 copolymer levels in
the presence of co-gellants

| MD 1648 (%) | Mp (° C.) | Hardness (mm) | Observation |
|---|---|---|---|
| 4^{a} | 103 | 209 | SH, S, SE |
| 5^{a} | 103 | 195 | H, S, SE |
| 4^{b} | 117 | 158 | H, S, SE |

For the evaluation of cone penetrometer hardness, a smaller number indicates a harder/firmer product. Thus, Table 8 shows that the resultant compositions became slightly harder/firmer as the copolymer level was increased from 4 to 5% and the product did not become too rubbery. Table 8 also demonstrates that increasing the concentration of co-gellant enables provides increased structuring of the gel stick compositions without making them too rubbery. However, all three blends with Kraton® MD 1648 were observed to be hazy.

### EXAMPLE 8

### Combination of Copolymers in Presence of Co-Gellant in the Compositions

The instant example evaluated production of a gel stick compositions comprising more than one block copolymer. In this regard, a concentration of 4% Kraton® G 1726 and a concentration of 1% Kraton® MD 6953 were incorporated into an oil blend comprising approximately 85% hydrogenated polyisobutene, 6% butyl stearate, 6% isostearyl alcohol, 0.25% GP-1, 0.25% EB-21, and 0.05% Tinogard. The resultant gel stick composition was observed to have an acceptable structure and also spread easily on the skin when tested. However, the resultant gel stick composition was observed to be hazy. The melting point of the gel stick composition was 103° C. and the penetrometer hardness was 160 mm. Accordingly, a blend of Kraton® G 1726 and Kraton® G 1643 ERS copolymers (e.g., 50:50) can produce a clear stick with desirable application (spreading) properties.

### EXAMPLE 9

### Reducing the Melting Points of Gel Stick Compositions

In some instances, it may be desirable to reduce the melting points of the gel stick compositions to about 100° C. or lower in order to simplify manufacturing. Of course, melting point parameters must be balanced with other properties of the gel stick compositions.

This example evaluates addition of the exemplary structuring agent isostearic acid into the blends to reduce the structure of the gelled stick and lower its melting point, while maintaining clarity of the resultant composition.

Approximately 3.5% isostearic acid and 4% Kraton® G 1726 were incorporated into an oil blend comprising approximately 85% hydrogenated polyisobutene, 6% butyl stearate, 6% isostearyl alcohol, 0.25% GP-1, 0.25% EB-21, and 0.05% Tinogard. The resultant gel stick composition was observed to have a softened structure, the ability to spread easily on the skin upon application, and a clear visual appearance. The melting point of the composition was 66° C. and its penetrometer hardness was 230 mm. Accordingly, melting point of gel stick compositions can be adjusted by controlling the levels of structuring agents (e.g., isostearic acid) that are introduced into the blends.

### EXAMPLE 10

### Creation of Co-Gellant Premixes

To simplify the making of oil gels from co-gellants, a co-gellant premix (80% Octyl dodecanol, 10% GP-1, and 10% EB-21) or other suitable combinations of solvents and co-gellants can be created. Co-gellant premixes can reduce the amount of time required to dissolve the co-gellants individually in oil blends. The co-gellant premix can simply be reheated by the user in order to liquefy it and then added to the rest of a blend in progress or used as the starting point of a blend. Table 3 and elsewhere shows that the co-gellant structure and level can be used to modify various properties of the gel stick composition such as melting point, clarity, and texture. The co-gellant premix can be an efficient vehicle for incorporation of these changes.

### EXAMPLE 11

### Process for Making Clear Gel Structured Bases

In this example, gel stick formulations comprising a clear gel structured base were formulated so that further compatible additives can be added. Many formulations of clear gel structured bases can accommodate up to approximately 30% of one or more additives. Table 9 provides an exemplary formula for a clear gel structured base utilizing hydrogenated polyisobutene. Similarly, structured clear gel structured bases can be prepared from mineral oil (M), C12-C15 Alkyl benzoate (ML), and Squalane (SQ).

TABLE 9
Clear gelled stick base (ME)

| Ingredient | W/W (%) |
|---|---|
| Hydrogenated Polyisobutene | 60.20 |
| Butyl stearate | 7.69 |
| Isostearyl alcohol | 7.69 |
| Kraton G 1726 | 5.13 |
| Octyl dodecanol | 15.38 |
| GP-1 (Dibutyl lauroyl glutamide) | 1.92 |
| EB-21 (Dibutyl ethylhexanoyl glutamide) | 1.92 |
| Tinogard | 0.06 |
| Total | 100.00 |

### EXAMPLE 12

### Process for Making Transparent Sunscreen Products

Various transparent products can be created from gel stick formulations comprising a clear gel structured base. The transparent products can be formulated by adding a suitable quantity of compatible additives to the clear gel structured bases.

The exemplary formula in Table 10 demonstrates use a clear gel structured base created from hydrogenated polyisobutene (ME) to formulate a transparent sunscreen product. Similar transparent sunscreen products can be prepared from mineral oil (M), C12-C15 Alkyl benzoate (ML), and Squalane (SQ) clear gel structured bases.

TABLE 11
Transparent sunscreen product created from clear gel
structured base (ME) and mixing procedure
Formula

| Ingredient | Phase | INCI | % w/w | Supplier |
|---|---|---|---|---|
| Versastique | A | Hydrogenated | 78.00 | Penreco |
| Clear ME 5T | | Polyisobutene, | | |
| | | Butyl Stearate, | | |
| | | Isostearyl Alcohol, | | |
| | | Hydrogenated | | |
| | | Styrene/Butadiene | | |
| | | Copolymer, Octyl | | |
| | | Dodecanol, | | |
| | | Dibutyl Lauroyl | | |
| | | Glutami de, | | |
| | | Dibutyl Ethylhexanoyl | | |
| | | Glutamide, | | |
| | | Pentaerythrityl | | |
| | | Tetra-Di-t-Butyl | | |
| | | Hydroxyhydrocinnamate | | |
| Avobenzone | B | Butyl Methoxydi | 3.00 | DSM |
| | | benzoyl methane | | |
| Homosalate | B | Homosalate | 10.00 | DSM |
| Octyl | B | Ethyl hexyl Salicylate | 5.00 | DSM |
| Salicylate | | | | |
| Octocrylene | B | Octocrylene | 4.00 | DSM |

Mixing Instructions
Heat phase A until completely dissolved (120 - 130° C.).
Start stirring and allow to cool to 115 C.
Add Avobenzone and stir until desolved.
Add the rest of phase B ingedients sequesntially with continued stirring.
Continue stirring at 115° C. for 1 hour.
Pour into desired packaging.

### EXAMPLE 13

### Process for Making Opaque Sunscreen Products

Various opaque products can be created from gel stick formulations comprising a clear gel structured base. The opaque products can be formulated by adding a suitable quantity of compatible additives to the clear gel structured bases.

The exemplary formula in Table 11 demonstrates use a clear gel structured base created from squalane to formulate an opaque sunscreen product. Similar opaque sunscreen products can be prepared from hydrogenated polyisobutene (ME) clear gel structured bases. Mineral sunscreen actives are currently perceived as having a superior safety profile than their organic counterparts.

TABLE 11
Opaque sunscreen stick created from clear gelled stick base (SQ)

| | | % | |
|---|---|---|---|
| Trade Name | INCI | w/w | Supplier |
| Versastique | Squalane, Butyl Stearate, | 78.0 | Penreco |
| Clear | Isostearyl Alcohol, | | |
| SQ 5T | Hydrogenated | | |
| | Styrene/Butadiene | | |
| | Copolymer, Octyl | | |
| | Dodecanol, Dibutyl | | |
| | Lauroyl Glutamide, | | |
| | Dibutyl Ethylhexanoyl | | |
| | Glutamide, | | |
| | Pentaerythrityl Tetra- | | |
| | Di-t-Butyl | | |
| | Hydroxyhydrocinnamate | | |
| Neossance | Zinc Oxide & C13-15 | 19.0 | Aprinnova |
| CleanScreen | Alkane & Polyglycery1-3 | | |
| Z80nano | polyricinoleate & | | |
| | Sorbitan Isostearate & | | |
| | Triethoxycaprylylsilane | | |
| Neossance | Titanium Dioxide & | 3.0 | Aprinnova |
| CleanScreen | C13-15 Alkane & | | |
| T65 | Polyglycery1-3 | | |
| | polyricinoleate | | |
| | & Sorbitan Isostearate & | | |
| | Silica & | | |
| | Triethoxycaprylylsilane | | |

The opaque sunscreen product can be formulated by heating phase 1 until it is completely dissolved (approximately 120-130° C.). Stirring is then commenced and the formulation is allowed to cool to about 120° C. Thereafter, the phase 2 ingredients are added sequentially with continued stirring. Stirring is continued at about 120° C. for approximately 1 hour. The formulation is then poured into desired packaging. The resultant product is observed to be a white solid stick that leaves no residue after application to the skin.

### EXAMPLE 14

### In Vitro Efficacy of Transparent Sunscreen Products

In the instant example, the in vitro SPF values for the transparent sunscreen products formulated with organic sunscreen agents were measured under both static/dry and dynamic/post-immersion conditions. Results are shown in Table 12.

TABLE 12
In vitro SPF performance of transparent organic sunscreen sticks

| Clear Gelled | | Post-immersion | UVA/UVB |
|---|---|---|---|
| Stick Base | Static SPF | SPF | ratio |
| CS SPF 50 WR | 61 | 47 | 0.90 |
| Mineral oil (M Clear) | 65 | 70 | 0.93 |
| Hydrogenated | 65 | 75 | 0.94 |
| polyisobutene | | | |
| (ME Clear) | | | |
| Squalane (SQ Clear) | 63 | 69 | 0.93 |

The transparent sunscreen products were formulated to deliver SPF 30 performance, so the static SPF of 61 to 65 for the three prototypes created from M, ME, and SQ Clear stick bases demonstrated a significant SPF boosting effect for these clear gelled stick bases. All three clear gelled stick bases showed higher post-immersion SPF compared to static/dry SPF, which is desirable. This result was unexpected, especially when compared to a gold standard traditional lotion-type sunscreen product formulated to deliver SPF 50 and water resistance (CS SPF50 WR).

The observed static/dry and post-immersion SPF performances evidence the unexpected advantages of the clear gelled stick bases formulated as sunscreen products. The broad-spectrum SPF performance observed in the clear gelled stick bases can be demonstrated by the UVA/UVB ratio of 90-94% for all of the sunscreen products evaluated in the instant example.

### EXAMPLE 15

### In Vitro Efficacy of Opaque Sunscreen Products

In the instant example, the in vitro SPF values for the opaque sunscreen products formulated with mineral sunscreen agents were measured under both static/dry and dynamic/post-immersion conditions. Results are shown in Table 13.

TABLE 13
In vitro SPF performance of opaque mineral sunscreen sticks

| Clear Gelled | | Post-immersion | UVA/UVB |
|---|---|---|---|
| Stick Base | Static SPF | SPF | ratio |
| Hydrogenated | 30 | 15 | 0.78 |
| polyisobutene | | | |
| (ME Clear) | | | |
| Squalane (SQ Clear) | 40 | 25 | 0.78 |

The opaque sunscreen products were formulated to deliver SPF 30 performance, so the static SPF of 40 for the prototype created from SQ Clear stick bases demonstrated a significant SPF boosting effect for these clear gelled stick bases. The post-immersion SPF shows superiority of the SQ Clear stick base by observing only 37% reduction vs. 50% reduction for the SQ Clear stick base. This result was unexpected.

The broad-spectrum SPF performance in the clear gelled stick bases is demonstrated by the observed UVA/UVB ratio of almost 80%.

### EXAMPLE 16

### Texture Analysis of Gel Stick Compositions

In this example, a texture analyzer was used to measure various properties of products of the instant disclosure. The products including the instant invention were compared to marketed products based on wax-thickened silicone, including Sun Burn SPF 30 (SB), Babyganics SPF 50 (BG), and Neutrogena Wet Skin Kids SPF 70+(NK). The formulated sunscreen products comprised Mineral Oil (M), Hydrogenated Polyisobutene (ME), and Squalane (SQ) gelled stick bases. Additives to the exemplary gelled stick bases included 22% of a sunscreen package (Avobenzone 3%, Homosalate 10%, Octyl salicylate 5%, and Octylcrylene 4%) to create the three exemplary sunscreen products: VS-M, VS-ME, and VS-SQ, respectively. Texture parameters including hardness, friction, and transfer rate were evaluated for the three marketed products (i.e., SB, BG, and NK) in comparison to the formulated exemplary sunscreen products (i.e., VS-M, VS-ME, and VS-SQ).

A. Hardness

Hardness was observed via evaluation of the maximum energy that was required to insert the probe into the tested sample. Hardness testing was performed using the texture analyzer shown in FIG. 8 with a needle penetrometer attachment. The setup and parameters for measuring hardness were as follows:
Test mode: Compression
Pre-test speed: 1 mm/s
Test speed: 1 mm/s
Post-test speed: 10 mm/s
Target mode: Distance
Force: 100 g
Distance: 5 mm
Strain: 10%
Trigger type: Auto (Force)
Trigger force: 1 g
Probe: PN-2
Points/s: 200

As shown in FIG. 9, the three marketed products comprising wax-thickened silicone (SB, BG, and NK) were observed to be significantly harder than the exemplary sunscreen products formulated according to the present disclosure (VS-M, VS-ME, and VS-SQ). The exemplary sunscreen products formulated were shown to have similar hardness properties.

B. Friction

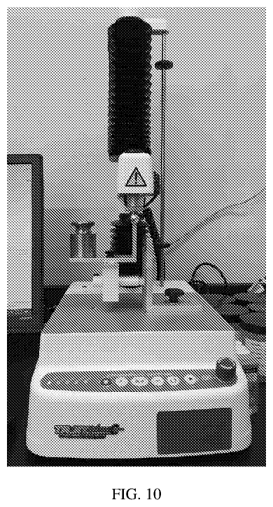

Friction was observed via evaluation of the force generated as the tested sample is spread over a surface. Hardness and friction were observed to be highly correlated. Friction testing was performed using the texture analyzer shown in FIG. 10. The setup and parameters for measuring friction were as follows:
Test mode: Compression
Pre-test speed: 6 mm/s
Test speed: 6 mm/s
Post-test speed: 6 mm/s
Target mode: Distance
Force: 500 g
Distance: 35 mm
Strain: 10%
Trigger type: Button
Trigger force: 5 g
Points/s: 200

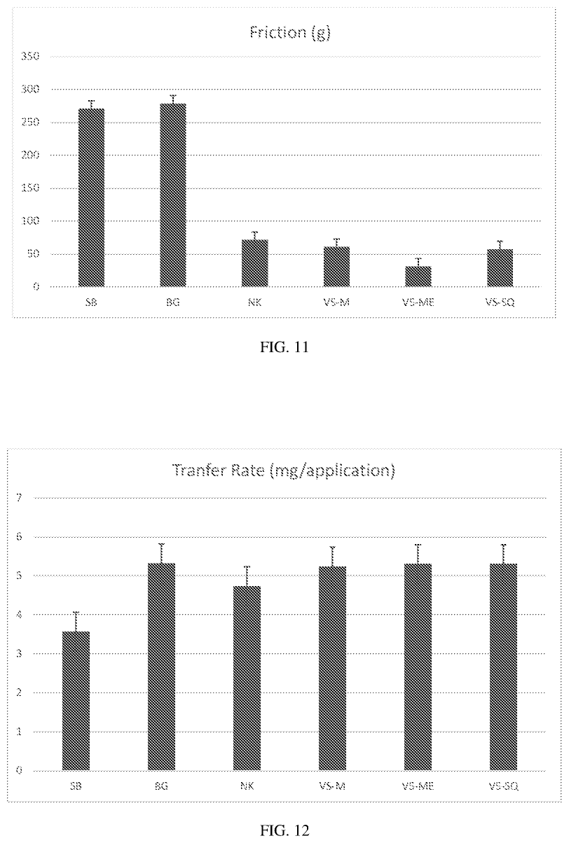

As shown in FIG. 11, the three marketed products comprising wax-thickened silicone (SB, BG, and NK) were observed to have significantly more friction than the exemplary sunscreen products formulated according to the present disclosure (VS-M, VS-ME, and VS-SQ). The exemplary sunscreen products formulated were shown to have similar friction properties, although VS-ME was slightly more lubricious.

C. Transfer Rate

Transfer rate was observed via evaluation of the amount of tested sample applied to the substrate under a constant application force. Hardness and friction were observed to not be reliable predictors of the transfer rate.

Transfer rate testing was performed using the texture analyzer shown in FIG. 10. The setup and parameters for measuring transfer rate were as follows:
Test mode: Compression
Pre-test speed: 6 mm/s
Test speed: 6 mm/s
Post-test speed: 6 mm/s
Target mode: Distance
Force: 500 g
Distance: 35 mm
Strain: 10%
Trigger type: Button
Trigger force: 5 g
Points/s: 200

As shown in FIG. 12, the three marketed products comprising wax-thickened silicone (SB, BG, and NK) had similar transfer rates compared to the exemplary sunscreen products formulated according to the present disclosure (VS-M, VS-ME, and VS-SQ). The exemplary sunscreen products formulated were shown to have similar transfer rates.

1. A gel stick composition comprising:
i) one or more emollients;
ii) one or more emulsifiers;
iii) one or more block copolymers;
iv) one or more co-gellants; and
v) one or more antioxidants.

2. (canceled)

3. (canceled)

4. (canceled)

5. (canceled)

6. (canceled)

7. The gel stick composition of claim 1, wherein the one or more emulsifiers is Uno Alkanol (Isostearyl alcohol).

8. The gel stick composition of claim 1, wherein the one or more block copolymers is selected from the group consisting of a diblock polymer, a triblock polymer, a star polymer, and combinations thereof.

9. The gel stick composition of claim 1, wherein the one or more block copolymers is selected from the group consisting of Kraton® G 1702, Kraton® G 1701, Kraton® G 1780, Kraton® G 1650, Kraton® G 1652, Kraton® D 1101, Kraton® D 1102, Kraton®D 1133, Kraton® G1901, Kraton® D1160, and combinations thereof.

10. The gel stick composition of claim 1, wherein the one or more block copolymers is selected from the group consisting of Kraton® G 1726, Kraton® G 1643 ERS, Kraton® G 1648, Kraton® MD 6953, and combinations thereof.

11. (canceled)

12. (canceled)

13. (canceled)

14. The gel stick composition of claim 1, wherein the one or more co-gellants is an amino acid dialkylamide.

15. The gel stick composition of claim 1, wherein the one or more co-gellants is present at about 0.01% to about 5% (w/w).

16. The gel stick composition of claim 1, wherein the one or more co-gellants is selected from the group consisting of GP-1 (dibutyl lauroylglutamide), EB-21 (dibutyl ethylhexanoyl glutamide), or a combination thereof.

17. (canceled)

18. (canceled)

19. The gel stick composition of claim 1 further comprising a clarifying component.

20. The gel stick composition of claim 19, wherein the clarifying component is selected from the group consisting of a structuring agent, a solubilizer, and a surfactant.

21. The gel stick composition of claim 19, wherein the clarifying component is a structuring agent.

22. (canceled)

23. (canceled)

24. (canceled)

25. The gel stick composition of claim 1 further comprising a processing component.

26. The gel stick composition of claim 25, wherein the processing component is selected from the group consisting of caprylic triglycerides and capric triglycerides.

27. A gel stick composition comprising:
i) a fatty alkane lipid, wherein the fatty alkane lipid is squalane;
ii) a fatty acid ester, wherein the fatty acid ester is Dermol BS (Butyl stearate);
iii) a fatty alcohol, wherein the fatty alcohol is Uno Alkanol (Isostearyl alcohol);
iv) a first block copolymer, wherein the first block copolymer is Kraton G 1650;
v) a second block copolymer, wherein the second block copolymer is Kraton G 1702;
vi) an amino acid dialkylamide co-gellant, wherein the amino acid dialkylamide co-gellant is GP-1 (dibutyl lauroylglutamide); and
vii) an antioxidant, wherein the antioxidant is Tinogard.

28. The gel stick composition of claim 27, wherein
i) the squalane is present at about 80.45% (w/w);
ii) the Dermol BS (Butyl stearate) is present at about 6% (w/w);
iii) the Uno Alkanol (Isostearyl alcohol) is present at about 6% (w/w);
iv) the Kraton G 1650 is present at about 5% (w/w);
v) the Kraton G 1702 is present at about 1.5% (w/w);
vi) the GP-1 (dibutyl lauroylglutamide) is present at about 1% (w/w); and
vii) the Tinogard is present at about 0.05% (w/w).

29. (canceled)

30. (canceled)

31. (canceled)

32. (canceled)

33. A gel stick composition comprising hydrogenated polyisobutene, butyl stearate, isostearyl alcohol, hydrogenated styrene/butadiene copolymer, octyl dodecanol, dibutyl lauroyl glutamide, dibutyl ethylhexanoyl glutamide, and pentaerythriyl tetra-di-t-butyl hydroxyhydrocinnamate.

34. The gel stick composition of claim 33 further comprising butyl methoxydibenzoylmethane.

35. The gel stick composition of claim 33 further comprising homosalate.

36. The gel stick composition of claim 33 further comprising ethylhexyl salicylate.

37. The gel stick composition of claim 33 further comprising octocrylene.

38.-40. (canceled)
